(19) United States
(12) Patent Application Publication
(10) Pub. No.: US 2011/0295184 A1
(43) Pub. Date: Dec. 1, 2011

(54) Preparation and Iontophoretic Device for the Transdermal Application of Active Ingredients

(76) Inventors: Andreas Koch, Melsbach (DE); Christoph Schmitz, Rheinbrohl (DE); Rolf Pracht, Hohr-Grenzhausen (DE)

(21) Appl. No.: 13/055,076

(22) PCT Filed: Jul. 8, 2009
(86) PCT No.: PCT/EP2009/004919

§ 371 Date: Jan. 20, 2011

(30) Foreign Application Priority Data
Jul. 21, 2008 (DE) 10 2008 034 098.7

(51) Int. Cl. A61N 1/30

(52) U.S. Cl. 604/20; 604/503

(57) ABSTRACT

The present invention relates to an iontophorectic device and a composition comprising active ingredients for the transdermal release of active ingredients, particularly of human insulin. Insulin, a blood sugar-lowering hormone inhibiting glycogen breakdown of the pancreas, can be made available as the most important pharmaceutical for the therapy of diabetes mellitus via the transdermal resorption route.

The present invention relates to a preparation and an iontophoresis device for the transdermal delivery of active ingredients, in particular human insulin.

Insulin, a hormone of the pancreas which lowers blood sugar and inhibits glycogen breakdown, is the most important medicament for treating the disease diabetes mellitus, a sugar-processing disorder due to relative or absolute lack of insulin with accompanying impairment of fat and protein metabolism as well as damage to the liver, cardiovascular system and nervous system.

Owing to its chemical nature, insulin is a peptide and can therefore be applied only via parenteral paths in which no proteolytic processes, which would inevitably lead to destruction of the insulin, take place. Administration via the gastrointestinal tract is unsuitable. Since its introduction, subcutaneous injection has become widespread as a preferred application path for insulin, inter alia because it can be carried out relatively economically and by the patient himself. Besides the infection risk, there are further problems for patients who suffer from so-called needle phobia. In the last decades, a search has therefore been made for other delivery options for insulin. Finally, in 2003, an insulin inhalation system was proposed for the pulmonary resorption path, but this system was withdrawn again from the market very soon after its introduction. Another path which has continually been the subject of much research work in recent years is the transdermal resorption route. Since the insulin molecule cannot cross the skin permeation barrier owing to its physicochemical properties, for example at 5700 daltons it has much too high a molecular weight, attempts have been made to bring insulin through the skin into the blood system by means of liposomal preparations in the form of so-called Transfersomes®. [1]

Transfersomes® are particular lipid vesicles, i.e. closed hollow spheres in the nanometer range, consisting of one or more lipid double layers, highly purified lecithin as a phospholipid forming the basic building block or basic backbone of the spherical membrane. Compared with their structurally related liposomes, Transfersomes® have a high membrane flexibility and therefore a strong liposome deformability, which seems to allow them even to penetrate into pores which are very much smaller than themselves. Since both the interiors of the spherical Transfersomes® and the interiors of the membranes themselves can be loaded with pharmaceutical substances, Transfersomes® as a kind of “shuttle” represent an interesting pharmaceutical presentation form for the dermal or transdermal transport route.

Whether liposomes or Transfersomes® actually lead to enhanced active ingredient dermal penetration compared with conventional pharmaceutical preparations, however, is highly disputed in the specialist literature and is also sometimes attacked with counterexamples. For instance, the authors in [2] have shown that in particular large hydrophilic molecules, such as the protein insulin, growth hormones or cyclosporin, cannot penetrate into the skin by means of Transfersomes®. Our own in vitro permeation studies with insulin, encapsulated in a transfersome-like preparation, could not produce any evidence for a “shuttle” effect; measurable traces of insulin (mass spectroscopy) could not be found either in the acceptor part under the skin (Franz diffusion cell) or in the skin itself. The statement by the authors in [1] should also be questioned since a human clinical study with liposomally prepared insulin for transdermal application, carried out by the Applicant Company, revealed a negative outcome. [3]

It was an object of the invention to overcome the disadvantages of the prior art and to make peptides or proteins, in particular large hydrophilic molecules, for example growth hormones or cyclosporin and in particular the protein insulin, available effectively but also economically in a way suitable for transdermal administration.

The object is achieved by a kit comprising an iontophoresis device and a preparation comprising active ingredients, wherein the preparation comprising active ingredients contains closed hollow bodies which are in the form of liposomes or micelles and contain an active ingredient from the group of peptides or proteins.

All the more surprisingly, in the present invention it has been possible to show that, for example, insulin encapsulated liposomally in a preparation can be made accessible to the transdermal resorption route by causing the liposomally encapsulated insulin to penetrate into the skin or permeate through the skin by means of an “electrical drive”, for example by iontophoresis, even though iontophoresis as the most effective method of transdermal permeation increase for charged pharmaceutical substances is precisely not applicable for insulin. [4]

The isoelectric point (isoelectric point=pH at which there is charge equilibrium, i.e. the concentration of the anion is equal to that of the cation of an ampholyte) of insulin lies at pH 5.4; i.e. at pH<5.4 the insulin molecule carries a positive charge (=cation) while at pH>5.4 it is negatively charged (=anion). Since there is a pH gradient in the skin, there being a pH of 5.2 on the skin surface which continuously approaches the physiological pH of 7.4 when moving deeper through the skin in the direction of the interior or blood vessels, the insulin molecule would inevitably change its state of charge in the course of a transdermal permeation route. The consequent result is a transport block of the transdermal passage, and the insulin would even migrate back again in the direction of the part of the active ingredient applied on the skin side.

Assuming that the pH in the insulin were set to 4.0, insulin would exist as a cation and could initially penetrate into the skin, provided that the active electrode over the insulin is connected as a positive electrode (=anode).

Owing to the pH gradient in the skin, however, the insulin molecule first becomes neutral since the isoelectric point lies at pH 5.4, and then actually becomes more and more negatively charged. The electromotive driving force of iontophoresis in the sense of permeation through the skin can therefore no longer work; on the contrary, according to the laws of electromigration the now negatively charged insulin would have to migrate back again in the direction of the positive electrode and therefore to the active ingredient reservoir.

In the converse case, i.e. the active electrode over the insulin is connected as a negative electrode (=cathode) and the pH of the insulin preparation is equal to or slightly >7 (skin irritation occurs beyond pH>8.5), so that the insulin is negatively charged, the migration or transport conditions are similar; the insulin molecule would already exist as a neutral molecule in the outermost skin layer, the stratum corneum, and not be able to penetrate further. For substance transport by means of iontophoresis, it is essential that the active ingredients to be transported are charged. In the case of peptides, the only ones suitable as transdermal candidates for iontophoresis are those whose isoelectric point lies at pH<4.0 or >8.0. [4]

In the case of the present invention, this problem is overcome in that on the one hand the liposomally encapsulated insulin does not experience any change in its charge conditions in the course of the transdermal penetration route, since it is encapsulated and therefore electrically shielded, and on the other hand it is probably not the electromigration of the iontophoresis (migration of charged particles toward their oppositely charged electrodes) which is responsible for the actual transport mechanism, but rather the so-called electro-osmosis, a process which occurs as an accompanying phenomenon or byproduct when applying an electric field to the skin. Owing to the differing water content in the individual skin layers, when an electric field is applied a potential gradient is formed which increases the permeability of the skin and leads to the formation of a liquid flow through the skin. This in turn is the reason why even uncharged, electrically neutral molecules, for example liposomal insulin, can penetrate to a greater extent into the skin during iontophoresis [5].

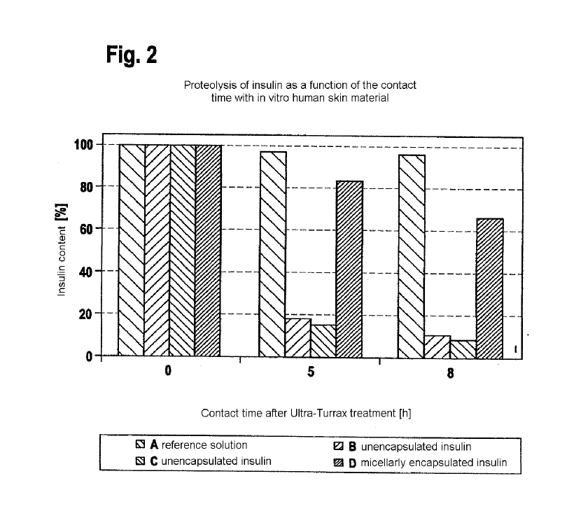

Merely using iontophoresis, the transdermal route of insulin or insulin analogs is also made very difficult and without therapeutic relevance [6] because proteolytic destruction reactions due to proteases occurring in the skin play a significant role. FIG. 2 shows for example the breakdown of insulin in a human skin sample, comminuted by means of an Ultra-Turrax treatment, under the effect of proteases belonging to the skin. After 8 hours, de facto no insulin can be detected any longer. It is therefore essential to protect insulin in the course of its transdermal resorption route, including in particular against attack by peptide-cleaving proteases. Liposomes are intended to mean spherical or ovoid structures consisting of one or several concentric lipid double layers with an aqueous interior, so-called lipid vesicles.

The shape of the liposomes is in the end unimportant for the subject-matter of the invention, and may differ significantly from the spherical shape. In the context of the present invention, liposomes and micelles are intended to mean hollow bodies which are capable of penetrating into the skin or permeating through the skin by means of iontophoresis. The diameter of the hollow bodies preferably lies in the range of from 25 nm to 1 μm. Owing to their stability, those with a diameter of from 100 to 300 nm are preferably used.

The Transfersomes® referred to above are likewise suitable in the context of this invention as a special preparation comprising active ingredients for transdermal administration and are included under the term liposomes in the application. For example, the production of Transfersomes® is specified in DE 44 47 287 C1, the disclosure content of which is part of this description. Liposomes are conventionally produced by suspending suitable lipids in aqueous solution. For example, phosphatidylcholines (lecithins), phosphatidylethanolamines or phosphatidylserines (kephalins) are used for this. Treating this mixture with ultrasound leads to a dispersion of approximately equally large closed liposomes. Such liposomes may, for example, also be produced by mixing an ethanol/lipid solution rapidly with water. If the lipid is injected into the aqueous solution through a thin needle, round liposomes with a diameter of approximately 50 nm are obtained.

Another method (the film method) consists in producing a homogeneous, transparent lipid film on the inner wall of a round-bottomed flask by means of a rotary evaporator. After the film has been dissolved in a suitable buffer solution, multilamellar liposomes of different sizes are spontaneously produced. If the resulting multilamellar liposome dispersion is extruded repeatedly under pressure, for example through a polycarbonate membrane with a defined pore size, uni- or oligolamellar liposomes are obtained which are characterized by a homogeneous size distribution. These methods are well known to the person skilled in the art.

It has furthermore surprisingly been possible to establish that insulin or insulin analogs can be made accessible to the transdermal resorption route by means of iontophoresis by being encapsulated through micelle formation with ionic or nonionic surfactants. Micelles are intended to mean the arrangement of individual molecules to form a larger unit, usually with a colloidal order of magnitude (association colloids), with a structure which is ordered owing to intermolecular forces.

Surfactant molecules in particular, for example fatty acids, bile acids, alkyl sulfates, fatty alcohol sulfates, fatty alcohol ether sulfates, sulfosuccinates, α-olefin sulfonates, isethionates, alkane and alkylbenzene sulfonates, saponins, quaternary ammonium salts, particularly preferably sodium dodecylsulfate or sodium cholate, may be envisaged as micelle-forming molecules.

Examples which may be envisaged for nonionic surfactants are polyethylene glycol ethers, phenol ethoxylates and alkylolamides, particularly preferably octylphenol-polyethylene glycol ether)₁₀. Micelle-forming molecules are distinguished in that they contain a hydrophobic hydrocarbon and a hydrophilic group in the molecule. In the case of forming spherical or rod-shaped micelles, other molecules may be incorporated owing to selective adsorption inside the associate being formed, and these can then be wetted as in the example of soaps, as oil droplets (lipophilic phase) for water (hydrophilic phase) and therefore be made miscible in water.

For effective micelle formation, the selected surfactants must be used in amounts which correspond to their so-called critical micelle formation. In the case of insulin or insulin analogs, on the one hand the charges in the molecule are covered or masked, so as to obviate the isoelectric point which is unfavorable for iontophoretic transport processes through human skin, and on the other hand the insulin molecule is protected by the outer protective envelope of the surfactant molecules surrounding it against proteolytic breakdown by proteases belonging to the skin.

The preparation comprising active ingredients is, for example, in the form of a solution, ointment, paste, foam or gel. Besides the hollow bodies containing active ingredients, it may contain other auxiliaries such as lipids, water, alcohols, gelling agents, emulsifiers, stabilizers and enhancers. The preparation may also contain proportions of hollow bodies, containing active ingredients, which have different active ingredients. In this way, for example, rapid-acting insulin or insulin analogs and insulin or insulin analogs with a long-term effect can be administered in one application.

Only by the combination of iontophoresis and liposomal and/or micelle-forming pharmacy can peptide(s) or protein(s), which exert the hormonal or hormone-like effect, in particular insulin or insulin analogs, be made available transdermally for a sufficient therapeutic application. According to the invention, at least 10 international units or at least 350 μg of insulin are preferably bioavailable. The content is determined either in vitro using the residual content (HPLC) in the skin after the end of permeation, or directly in vivo using plasma determination or determination of the blood sugar level.

The preparation ideally still has at least 50% of its initial active ingredient content during its transdermal passage over a period of 5 hours. Here, the content is again determined in vitro using the residual content (HPLC) in the skin after prior extraction, in vitro representing a model for in vivo.

Besides the transdermal administration of insulin or insulin analogs, the invention is likewise suitable for the administration of physiologically highly active peptides which exert a hormonal or hormone-like effect (peptide hormones), including their derivatives or conjugates with an average molecular weight Mw of from 300 to 1,000,000 (daltons). In general peptide hormones are oligo-, and even more frequently polypeptides (having up to 100 amino acids), and sometimes actually higher molecular weight proteins (proteohormones). The peptide and protein active ingredients may be used as free acids or bases.

The active ingredient may have an average molecular weight of less than 3000 Da. Examples of such active ingredients are in particular abarelix, angiotensin II, anidulafungin, antide, argipressin, azalin and azalin B, bombesin antagonist, bradykinin, buserelin, cetrorelix, cyclosporin A, desmopressin, detirelix, enkephalins (leu-, met-) ganirelix, gonadorelin, goserelin, growth hormone secretagogue, micafungin, nafarelin, leuprolide, leuprorelin, octreotid, orntide, oxytocin, ramorelix, secretin, somatotropin, terlipressin, tetracosactide, teverelix, triptorelin, thyroliberin, thyrotropin, vasopressin.

The active ingredient may have an average molecular weight of from 3000 to 10,000 Da. Examples of such active ingredients are in particular calcitonin, corticotropin, endorphins, epithelial growth factor, glucagon, insulin, novolin, parathyroid hormone, relaxin, pro-somatostatin, salmon secretin.

The active ingredient may have an average molecular weight of more than 10,000. Examples of such active ingredients are in particular interferons (alpha, beta, gamma), interleukins (IL1, IL2), somatotropin, erytropoietin, tumor necrosis factor (TNF alpha, beta), relaxin, endorphin, dornase alpha, follicle stimulating hormone (FSH), human chorionic gonadotropin (HCG), human growth hormone release factor (hGRF), luteinizing hormone (LH) or epidermal growth factor.

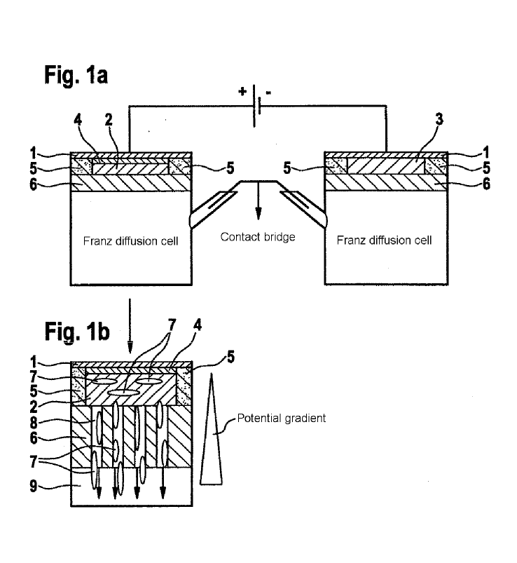

The invention will be further explained below with the aid of Table 1, FIGS. 1-4 and Examples 0-4.

Table 1 contains a summary of all the in vitro permeation results.

The in vitro permeation measurements of the systems according to the invention were carried out on the human full-skin in vitro skin model (FIGS. 1a and 1b) with the aid of modified Franz diffusion cells, configured as a representation of iontophoresis. The acceptor medium used in all cases was 0.025 molar HEPES buffer solution (HEPES: 2-[4-(2-hydroxyethyl)-1-piperazinyl]ethanesulfonic acid), adjusted to pH 7.4 and thermostatted at 32° C.

FIG. 1a represents the in vitro experimental arrangement for the transdermal application of human insulin by means of the combined use of iontophoresis and liposomal or micellar pharmacy. FIG. 1b represents the effect of electro-osmosis, produced during the application.

The references are:

(1) Ag/AgCl electrode as anode and cathode (active electrode and back electrode).
(2) verum compartment of anode or cathode, in the form of a liposome or micelle.
(3) electrolyte compartment of anode or cathode, active ingredient-free.
(4) electrolyte layer, as conductivity enhancer for the verum part in the anode or cathode.
(5) self-adhesive bonding ring with application fleece for active ingredient takeup.
(6) upper part of the human skin (epidermis with non-predamaged stratum corneum).
(7) liposomes or micelles, electrically neutral or electrically charged negatively, containing human insulin.
(8) liquid flow resulting from electro-osmosis, a side effect of iontophoresis, with the aid of which the liposomes containing insulin can for the first time migrate through, or permeate.
(9) dermis with blood capillary vessels for systematically transporting away the transdermally applied active ingredient.

The current source used was conventionally a DC generator (Hameg HM 7042-5, from Hameg in Mainhausen, Germany), which was adjusted to a constant current strength output of 500 μA/cm² of skin resorption surface. Full-surface Ag/AgCl electrodes from NAImco, Chattanooga (USA) were used as electrode material. The electrolyte reservoir of the back electrode (cathode or anode) consisted of a 2% strength solution of hydroxypropyl cellulose, supplemented with 0.9 wt % NaCl, which was applied with a surface density of 3 g/30 cm² onto a fleece, consisting of nonwoven polyester fleece (Paramol N 260-300P, from Lohmann & Rauscher, Neuwied, Germany). Approximately 200 mg of the corresponding insulin preparation were applied onto the identical fleece of the back electrode in the anode or cathode reservoir, the configuration as an anode or cathode being dictated by the charge of the corresponding hollow body. For the purpose of fixing on the skin, the edge consisted of a self-adhesive foam ring made of polyolefins (3M, type 1779). The permeation time, or iontophoresis treatment, was 5 hours in all the experiments; in the case of the Transfersomes, the polarity of the electrodes was respectively reversed after 2.5 h. The contact between the cells was established by a silver wire as a bridge.

After the end of permeation, the skin was subjected to a residual insulin content determination by initially cutting it into a plurality of small parts using scissors, then extracting for 5 hours while shaking in hydrochloric acid/70% ethanol, in order subsequently to examine it for insulin by means of a specific HPLC method. In order to show that insulin had in fact crossed the skin permeation barrier of the human skin, the stratum corneum, the skin was prepared for the residual content determination by removing the stratum corneum by means of so-called “tape stripping” [7] before the extraction of insulin from the skin sample. In order to highlight the selectivity of the analytical method used and the validity of the detected insulin, the in vitro permeation experiments were respectively carried out with verum and placebo samples (the same pharmaceutical preparation without any active ingredient).

FIG. 2 shows the proteolytic breakdown process of isolated insulin, i.e. the insulin is not liposomally or micellarly encapsulated, in comparison with the much lower breakdown of insulin which is protected against proteases by liposome or micelle formation.

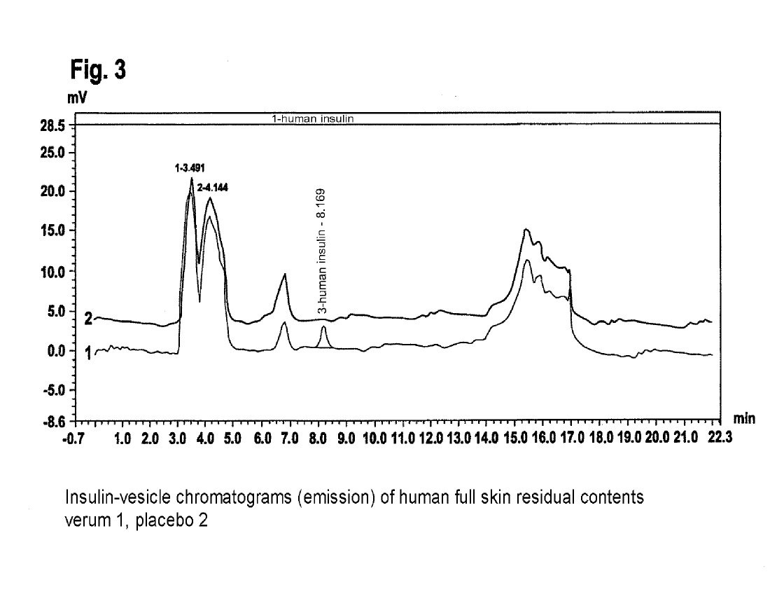

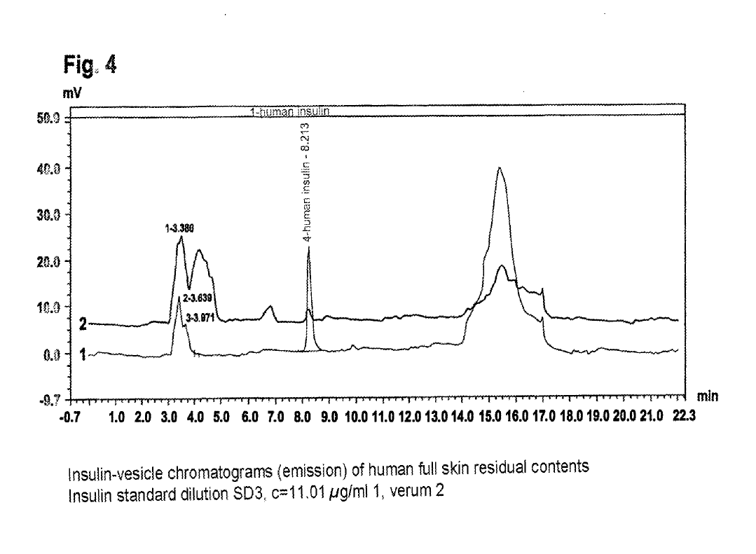

FIGS. 3 and 4 show by way of example HPLC chromatograms of verum and placebo samples from the permeation studies for the insulin preparations, here for Inventive Example 1. No peak within the retention time range of insulin can be seen in the placebo sample; the peak identified as insulin is therefore actually insulin, which is additionally confirmed by the chromatograms in FIG. 4 (HPLC chromatogram of the corresponding insulin standard sample) and UV spectral comparison of the standard and verum samples (not shown as a figure here). The same could be demonstrated for the other Inventive Examples, for which reason it is only mentioned once by way of example.

FIG. 3 shows insulin-liposome chromatograms (emission) of human full skin residual contents, dissolved in 0.01 M hydrochloric acid/70% ethanol, the verum (1) and placebo (2) being represented.

FIG. 4 shows insulin-standard and insulin-liposome chromatograms (emission) of human full skin residual contents, dissolved in 0.01 M hydrochloric acid/70% ethanol, the insulin standard dilution SD3, c=11.01 μg/ml (1) and verum (2) being represented.

TABLE 1
Result overview of insulin iontophoresis experiments with different pharmaceutical
preparations which respectively cover or mask the insulin's own charge

| | | | | | Size [cm²] for a 350 μg |
|---|---|---|---|---|---|
| | Vehicle | Amount | Skin | | amount delivered |
| Inventive | (micelle | applied | content | % | (corresponds to basal |
| example | former) | [μg/cm²] | [μg/cm²] | permeated | bolus therapy) |
| 0 | Only buffer I¹ | 1680 | No insulin | | |
| (comparative | (Tris/HCl) | | detectable | | |
| example) | | | | | |
| 1 | bile acid I¹ | 1598 | 6.4 | 0.40 | 55 |
| 2 | Na | 1680 | 13.3 | 0.80 | 26 |
| | dodecylsulfate | | | | |
| | I¹ | | | | |
| 3 | Triton X-100 | 26.5 | 3.53 | 13 | 85 |
| | I² | | | | |
| 4 | liposome I² | 500 | 1.90 | 0.38 | 184 |

I¹ - connection of the verum compartment as the cathode since the vehicle externally carries a negative charge.
I² - connection of the verum compartment first as the cathode then repoling as the anode after 2.5 h, since the vehicle is externally electroneutral.

The results show that at least for Inventive Examples 1-3, it is possible to carry out transdermal therapy for type 1 diabetes mellitus with acceptable TTS sizes <100 cm². Particularly for the treatment of a chronic metabolic disorder (diabetes mellitus), which is due to a lack of insulin or reduced insulin effect, treatment can be performed outstandingly with the subject-matter of the invention.

The method for the transdermal administration of insulin or insulin analogs is characterized by the following steps:
a) producing a preparation comprising active ingredients containing insulin or insulin analogs in closed hollow bodies, the hollow bodies being in the form of liposomes or micelles
b) applying the preparation together with the iontophoresis device onto the skin
c) carrying out the iontophoresis.

FIG. 2 shows the proteolysis of insulin as a function of the contact time with in vitro human skin material. The influence of proteases belonging to the skin on the breakdown of insulin after an insulin buffer solution with a defined content, together with in vitro human skin patches (24 cm²) which were comminuted by means of an Ultra-Turrax dispersing device, had been in contact over a period of 8 h by stirring, can be seen clearly. The breakdown in the micellar insulin sample (Triton X-100® as a micelle-forming nonionic surfactant) takes place much more slowly; here, the insulin content is still much more than 50% even after 8 hours of contact time. In this figure:

A denotes the reference solution
B, C denote unencapsulated insulin
D denotes micellarly encapsulated insulin

The same system can in principle be used as a technical embodiment in practice, as described above for the in vitro permeation or resorption studies. For example, full-surface Ag/AgCl electrodes from NAImco, Chattanooga (USA) which are selectively available in various sizes (1.5-4 cm²), are self-adhesive and are provided with a polyester fleece for takeup of the active ingredient preparations, are used as electrode material. The preparation comprising active ingredients is placed in either the anodic or cathodic part of the iontophoresis device.

The electrolyte reservoir of the back electrode (cathode or anode) consists, for example, again of a 2% strength solution of hydroxypropyl cellulose, supplemented with 0.9 wt % of sodium chloride, which is applied with a surface density of 3 g/30 cm² into/onto the application fleece of the NAImco® electrodes. For example, the corresponding electrodes [“dispersive” (return) electrode] of the iontophoresis application kit (“ionto+plus HI-Performance”) from NAImco may also be used as back electrodes. These are already provided with a conductive and self-adhesive polymer containing buffer. The permeation time or iontophoresis treatment should preferably not exceed a period of 5 hours. Preferably, the polarity of the electrodes is reversed after half the duration of the iontophoresis, if hollow bodies which are electrically neutral externally are used. In the case of using liposomes and micelles with non-ionic surfactants, the electrodes are therefore respectively repoled after 2.5 hours in a 5 hour application.

As a current supply source with regulation or control of the current strength, a corresponding device likewise from NAImco is used: type reference “id³ drug delivery device” which, owing to its size, is fixed by means of hook-and-loop fastening for example on the upper arm or above the wrist. The electrodes are connected to the current generator by means of conventional cables with a banana jack connection.

### INVENTIVE EXAMPLE 1

### Production of a Micellar Preparation by Means of Sodium Oxycholate

First, a Tris/HCl buffer/glycerol/water mixture (pH 6.8) with the following composition is prepared (Tris: 2-amino-2-(hydroxymethyl)propane-1,3-diol or amino-tris(hydroxymethyl)methane).

25.0 ml 0.5 M Tris/1 M HCl

11.5 ml glycerol

63.5 ml water

366 mg of sodium oxycholate, corresponding to 8.5 mmol, are then introduced into this mixture and stirred, until a clear solution is obtained (Solution A). Approximately 100 mg of human insulin are subsequently weighed into a 10 ml measuring flask, and filled up with Solution A. The batch is stirred at room temperature until full dissolving of the insulin, or for at least one hour, and then directly used for the permeation experiments.

### INVENTIVE EXAMPLE 2

### Production of a Micellar Preparation by Means of Sodium Dodecylsulfate

The production is carried out similarly as in Inventive Example 1, with the difference that 2 g of sodium dodecylsulfate are introduced instead of sodium oxycholate. The batch is again stirred at room temperature until full dissolving of the insulin, or for at least one hour, and then directly used for the permeation experiments.

### INVENTIVE EXAMPLE 3

### Production of a Micellar Preparation by Means of Triton X-100®

The production is carried out similarly as in Inventive Example 1, with the difference that 323.5 g of Triton X-100® (corresponding to 5 mmol or 0.3%) are introduced instead of sodium oxycholate, in which case the Triton X-100® must be provided first, after which it is filled up with the buffer-glycerol mixture.

Furthermore, instead of 100 mg of human insulin, only 15.4 mg are weighed in a 100 ml measuring flask and filled up with the buffer-glycerol mixture containing Triton X-100® (Triton X-100®=octylphenol-poly(ethylene glycol ether)₁₀). The batch is again stirred at room temperature until full dissolving of the insulin, or for at least one hour, and then directly used for the permeation experiments.

### INVENTIVE EXAMPLE 4

### Production of a Liposomal Preparation by Means of L-α-Phosphatidylcholine According to the Film Method [8]

First, 187.2 mg of L-α-phosphatidylcholine (lecithin) are dissolved in 10 ml of methanol. 2.5 ml of this lipid stock solution are subsequently transferred by means of an Eppendorf pipette into a 50 ml round-bottomed flask, then the tip is washed a further two times with 2.5 ml of methanol in each case. Extracting the methanol in a rotary evaporator (40° C., rotation level 4-5, no methanol smell should still be perceptible) leads to a transparent lipid film which is dried further in a high vacuum (p=0.05 mbar) for 2 hours in order to remove any solvent residues. 2 ml of a buffer solution, made up as follows and containing 0.1% insulin, are then pipetted into the round-bottomed flask:

10 mmol HEPES and 150 mmol NaCl, adjusted with 1 N NaOH to pH 7.4

5 mg of human insulin are dissolved in 5 ml of this buffer solution while stirring for one hour. The round-bottomed flask with the buffer solution containing insulin is shaken overnight at 240 revolutions per minute on a shaking machine.

### COMPARATIVE EXAMPLE 0

### Production of a Buffer Solution Containing Insulin without Micelle and/or Liposome Formation

The production is carried out similarly as in Inventive Example 1, with the difference that no sodium oxycholate is introduced. The batch is again stirred at room temperature until full dissolving of the insulin.

From all the batches (Comparative Example 0 and Inventive Examples 1-4), corresponding preparations were produced without human insulin (placebo batches) and were tested together with the respective verum batches in the iontophoretic in vitro permeation experiments (as an insulin negative control or evidence of the selectivity of the analytical HPLC method used for determining the residual insulin content in the skin).

### LIST OF SOURCES

[1] G. Cevc/Drug Delivery across the skin. Exp. Opin. Invest. Drugs 6, p. 1887-1937 (1997)
[2] H. Schreier, J. Bouwstra/Liposomes and niosomes as topical drug carriers: dermal and transdermal drug delivery. J. Contr. Rel. 30, p. 1-155 (1994)
[3] LTS Clinical Study Eutra CT Nr.: 2004-004043-21
[4] Y. N. Kalia et al./Advanced Drug Delivery Reviews 56 (2004) 619-658
[5] R. R. Burnette/Iontophoresis, in: Transdermal Drug Delivery—Developmental Issues and Research Initiatives, (Eds.) J. Hadgraft and R. H. Guy, Marcel Dekker, New York, 247-292 (1989)
[6] L. Langkjaar et al./Iontophoresis of monomeric insulin analogues in vitro: Effects of insulin charge across skin pre-treatment. J. controlled Release 51: p. 42-56 (1998)
[7] C. Herkenne et al./In vivo Methods for the Assessment of Topical Drug Bioavailability: Pharm. Res., Vol. 25, No. 1, p. 87-103 (2008)
[8] A. Bangham/Diffusion of Univalent ions across the lamellae of swollen phospholipids: J Mol Biol 13(1): 238-252 (1965)

1-19. (canceled)

20. A kit comprising an iontophoresis device and a preparation comprising active ingredients, wherein the preparation comprising active ingredients contains closed hollow bodies which are in the form of liposomes or micelles and contain an active ingredient from the group of peptides or proteins.

21. A kit as claimed in claim 20, wherein the preparation comprising active ingredients is in the form of a solution, ointment, paste, foam or gel.

22. A kit as claimed in claim 20, wherein the active ingredient in the preparation is a peptide hormone or a proteohormone.

23. A kit as claimed in claim 20, wherein at least 10 international units or at least 350 μg of insulin are bioavailable.

24. A kit as claimed in claim 20, wherein the content of active ingredient(s) in the preparation still comprises at least 50% of its initial content during its transdermal passage over a period of 5 hours.

25. A kit as claimed in claim 20, wherein the preparation comprising active ingredients is placed in the anodic or cathodic part of the iontophoretic device.

26. A kit as claimed in claim 20, wherein the polarity of the electrodes is reversed after half the duration of the iontophoresis.

27. A kit as claimed in claim 20, wherein the active electrode and the back electrode each consist of foils, which are coated with a mixture of silver and silver chloride.

28. A kit as claimed in claim 20, wherein the active electrode and the back electrode are configured as self-adhesive systems.

29. A kit as claimed in claim 20, wherein the micelles are made of ionic surfactants.

30. A kit as claimed in claim 29, wherein the ionic surfactant is selected from the group consisting of fatty acids, bile acids, alkyl sulfates, fatty alcohol sulfate, fatty alcohol ether sulfates, sulfosuccinates, α-olefin sulfonates, isethionates, alkane and alkylbenzene sulfonates, and saponins.

31. A kit as claimed in claim 30, wherein the ionic surfactant is a sodium dodecylsulfate or sodium cholate.

32. A kit as claimed in claim 20, wherein the micelles are made of nonionic surfactants.

33. A kit as claimed in claim 32, wherein the nonionic surfactant used is selected from the group consisting polyethylene glycol ethers, phenol ethoxylates and alkylolamides.

34. A kit as claimed in claim 33, wherein the nonionic surfactant is octylphenol-poly(ethylene glycol ether)₁₀.

35. A kit as claimed in claim 20, wherein the liposomes are made of lipids selected from the group comprising phosphatidylcholine, phosphatidylethanolamine or phosphatidylserine.

36. A method for the transdermal administration of insulin or insulin analogs comprising:
a. producing a preparation comprising active ingredients containing insulin or insulin analogs in closed hollow bodies, the hollow bodies being in the form of liposomes or micelles;
b. applying the preparation together with the iontophoresis device onto the skin; and
c. carrying out the iontophoresis.

37. The method for transdermal administration of claim 36, wherein the polarity of the electrodes is reversed after half the duration of the iontophoresis.

38. A method for the treatment of diabetes mellitus due to the lack of insulin or a reduced insulin effect which comprises of administering the kit to a patient in need thereof.

39. A kit as claimed in claim 31, wherein
the preparation comprising active ingredients is in the form of a solution, ointment, paste, foam or gel;
the active ingredient in the preparation is insulin or an insulin analog, wherein at least 10 international units or at least 350 μg of insulin are bioavailable;
the content of active ingredient(s) in the preparation still comprises at least 50% of its initial content during its transdermal passage over a period of 5 hours;
the preparation comprising active ingredients is placed in the anodic or cathodic part of the iontophoretic device;
the polarity of the electrodes is reversed after half the duration of the iontophoresis;
the active electrode and the back electrode each consist of foils, which are coated with a mixture of silver and silver chloride; and the active electrode and the back electrode are configured as self-adhesive systems.

40. A kit as claimed in claim 34, wherein
the preparation comprising active ingredients is in the form of a solution, ointment, paste, foam or gel;
the active ingredient in the preparation is insulin or an insulin analog, wherein at least 10 international units or at least 350 μg of insulin are bioavailable;
the content of active ingredient(s) in the preparation still comprises at least 50% of its initial content during its transdermal passage over a period of 5 hours;
the preparation comprising active ingredients is placed in the anodic or cathodic part of the iontophoretic device;
the polarity of the electrodes is reversed after half the duration of the iontophoresis;
the active electrode and the back electrode each consist of foils, which are coated with a mixture of silver and silver chloride; and the active electrode and the back electrode are configured as self-adhesive systems.
